(19) United States
(12) Patent Application Publication
(10) Pub. No.: US 2024/0307340 A1
(43) Pub. Date: Sep. 19, 2024

(54) IMMUNOSUPPRESSIVE PHARMACEUTICAL COMPOSITION INCLUDING BENZENE DERIVATIVE AS IMMUNOSUPPRESSANT

(71) Applicant: INJE UNIVERSITY INDUSTRY-ACADEMIC COOPERATION FOUNDATION, Gimhae-si, Gyeongsangnam-do (KR)

(72) Inventor: Seok Ju PARK, Busan (KR)

(73) Assignee: INJE UNIVERSITY INDUSTRY-ACADEMIC COOPERATION FOUNDATION, Gimhae-si, Gyeongsangnam-do (KR)

(21) Appl. No.: 18/263,059

(22) PCT Filed: Jan. 19, 2022
(86) PCT No.: PCT/KR2022/001000

§ 371 Date: Jul. 26, 2023

(30) Foreign Application Priority Data
Jan. 29, 2021 (KR) 10-2021-0013723

(51) Int. Cl. A61K 31/235; A61K 31/166; A61P 17/06; A61P 35/00; A61P 37/06

(52) U.S. Cl. CPC A61K 31/235; A61K 31/166; A61P 17/06; A61P 35/00; A61P 37/06

(57) ABSTRACT

The present invention discloses the use of benzene derivatives as immunosuppressants. The benzene derivatives as immunosuppressants are represented by Formula 1:

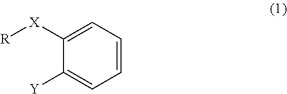

wherein R is a linear alkyl group, a branched alkyl group or an alkyl group containing one or more carbon-carbon double bonds, X is an amide or ester group, and Y is a halo, methoxy, ethoxy, hydroxyl or nitro group. The benzene derivatives are used in the preparation of immunosuppressive medicaments. The benzene derivatives are effective for treating T cell-mediated immune diseases, for example, for preventing and treating rejection after organ and bone marrow transplantation and treating T cell-mediated autoimmune diseases, psoriasis, multiple sclerosis, rheumatoid arthritis, insulin-dependent diabetes mellitus, specific hemolytic anemia, ulcerative colitis, myasthenia gastrica, glomerulonephritis, Guillain-Barre syndrome, and allergic diseases such as allergic asthma and atopy due to their ability to inhibit the proliferation of T lymphocytes.

### TECHNICAL FIELD

The present invention relates to the use of benzene derivatives as immunosuppressants, and more specifically to the use of benzene derivatives as immunosuppressants that are effective for treating T cell-mediated immune diseases, for example, for preventing and treating rejection after organ and bone marrow transplantation and treating T cell-mediated autoimmune diseases, psoriasis, multiple sclerosis, rheumatoid arthritis, insulin-dependent diabetes mellitus, specific hemolytic anemia, ulcerative colitis, myasthenia gastrica, glomerulonephritis, Guillain-Barre syndrome, and allergic diseases such as allergic asthma and atopy due to their ability to inhibit the proliferation of T lymphocytes.

### BACKGROUND ART

Immunity is an important phenomenon necessary to protect the body against antigens such as pathogens.

Immune responses are mediated by early innate and late adaptive immune responses.

Innate immunity, also called native immunity, non-specific immunity, primary defense action or natural immunity, refers to an immune system that does not recognize and responds immediately to specific pathogens.

Unlike innate immunity, adaptive immunity has exquisite specificities for different macromolecules and may cause very strong immune responses upon repeated exposure to the same antigens.

The two types of adaptive immune responses are cell-mediated immunity and humoral immunity. Cell-mediated immunity is mediated by T lymphocytes.

T lymphocytes are divided into functionally distinct populations: helper CD4 (helper) T cells, including Th1 cells, Th2 cells, Th17 cells, and Treg cells; and CD8 cytotoxic T lymphocytes.

T cells recognize Class I and Class II MHC molecules in grafts with different genetic backgrounds after tissue or organ transplantation and cause specific immune responses, called rejection responses, that destroy the grafts. For successful organ or tissue transplantation, T cell-mediated immune responses need to be regulated.

For allergic diseases, some CD4+ helper T cells release IL-4 and IL-13 that stimulate isotype conversion of B-cell antibodies to immunoglobulin E (IgE). These T cells play a critical role in producing IgE, a special type of antibody that is very important in the pathogenesis of atopy. Thus, it is very important to regulate the immune responses of T cells.

Autoimmune diseases refer to diseases in which the immune system against foreign antigens such as pathogens recognizes its own organs or tissues as antigens of foreign origin to cause immune responses. T cells are known to be involved in the pathogenesis of autoimmune diseases. Such autoimmune diseases include rheumatoid arthritis, multiple sclerosis, inflammatory bowel disease, and psoriasis, whose mechanisms are known to be inflammation mediated by T-cell cytokines.

For the treatment of these diseases, it is also very important to regulate the immune responses of T cells. Therefore, inhibition of T cell proliferation is of great importance to regulate the immune responses of T cells.

### PRIOR ART DOCUMENTS

### Patent Documents

(Patent Document 0001) Korean Patent Publication No. 10-2008-0013886 (published on Feb. 13, 2008)

### DISCLOSURE OF INVENTION

### Technical Problem

Therefore, an object of the present invention is to provide the use of benzene derivatives as immunosuppressants that are effective for treating T cell-mediated immune diseases, for example, for preventing and treating rejection after organ and bone marrow transplantation and treating T cell-mediated autoimmune diseases, psoriasis, multiple sclerosis, rheumatoid arthritis, insulin-dependent diabetes mellitus, specific hemolytic anemia, ulcerative colitis, myasthenia gastrica, glomerulonephritis, Guillain-Barre syndrome, and allergic diseases such as allergic asthma and atopy due to their ability to inhibit the proliferation of T lymphocytes.

### Solution to Problem

An aspect of the present invention provides the use of a compound represented by Formula 1:

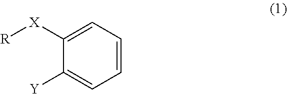

wherein R is a linear alkyl group, a branched alkyl group or an alkyl group containing one or more carbon-carbon double bonds, X is an amide or ester group, and Y is a halo, methoxy, ethoxy, hydroxyl or nitro group, in the preparation of an immunosuppressive medicament.

According to one embodiment of the present invention, X and Y in Formula 1 may be in the ortho position.

According to a further embodiment of the present invention, R in Formula 1 may have 6 to 14 carbon atoms.

According to another embodiment of the present invention, the medicament may be useful for inhibiting cell-mediated immunity.

According to another embodiment of the present invention, the medicament may be useful for the treatment of allograft rejection.

According to another embodiment of the present invention, the medicament may be useful for the treatment of autoimmune diseases.

According to another embodiment of the present invention, the medicament may be useful for the treatment of cancer diseases.

According to another embodiment of the present invention, the medicament may be useful for the treatment of psoriasis.

According to another embodiment of the present invention, the medicament may be useful for the treatment of atopy.

According to another embodiment of the present invention, the medicament may be useful for the treatment of insulin-dependent diabetes mellitus.

According to another embodiment of the present invention, the medicament may be useful for the treatment of multiple sclerosis.

According to another embodiment of the present invention, the medicament may be useful for the treatment of rheumatoid arthritis.

### Advantageous Effects of Invention

According to the present invention, the benzene derivative of Formula 1 can inhibit the proliferation of T lymphocytes, thus being effective as an immunosuppressant. Due to this ability, the benzene derivative can be used to treat T cell-mediated immune diseases. For example, the benzene derivative can be used to prevent and treat rejection after organ and bone marrow transplantation and treat T cell-mediated autoimmune diseases, psoriasis, multiple sclerosis, rheumatoid arthritis, insulin-dependent diabetes mellitus, specific hemolytic anemia, ulcerative colitis, myasthenia gastrica, glomerulonephritis, Guillain-Barre syndrome, and allergic diseases such as allergic asthma and atopy.

### BRIEF DESCRIPTION OF DRAWINGS

FIG. 1 shows the degrees of inhibition of T cell division by different concentrations of the compound of Example 1.

FIG. 2 shows the degrees of inhibition of T cell division by different concentrations of the compound of Example 2.

FIG. 3 shows the degrees of inhibition of T cell division by different concentrations of the compound of Example 3.

FIG. 4 shows the degrees of inhibition of T cell division by different concentrations of the compound of Example 4.

FIG. 5 shows the degrees of inhibition of T cell division by different concentrations of the compound of Example 5.

FIG. 6 shows the degrees of inhibition of T cell division by different concentrations of the compound of Example 6.

FIG. 7 shows the degrees of inhibition of T cell division by different concentrations of the compound of Example 7.

FIG. 8 shows the degrees of inhibition of T cell division by different concentrations of the compound of Example 8.

FIG. 9 shows the degrees of inhibition of T cell division by different concentrations of the compound of Example 9.

FIG. 10 shows the degrees of inhibition of T cell division by different concentrations of the compound of Example 10.

FIG. 11 shows the degrees of inhibition of T cell division by different concentrations of the compound of Example 11.

FIG. 12 shows the degrees of inhibition of T cell division by different concentrations of the compound of Example 12.

FIG. 13 shows the degrees of inhibition of T cell division by different concentrations of the compound of Example 13.

FIG. 14 shows the degrees of inhibition of T cell division by different concentrations of the compound of Example 14.

FIG. 15 shows the degrees of inhibition of T cell division by different concentrations of the compound of Example 15.

FIG. 16 shows the degrees of inhibition of T cell division by different concentrations of the compound of Example 16.

FIG. 17 shows the degrees of inhibition of T cell division by different concentrations of the compound of Example 17.

FIG. 18 shows the degrees of inhibition of T cell division by different concentrations of the compound of Example 18.

FIG. 19 shows the degrees of inhibition of T cell division by different concentrations of the compound of Example 19.

FIG. 20 shows the degrees of inhibition of T cell division by different concentrations of the compound of Comparative Example 1.

FIG. 21 shows the degrees of inhibition of T cell division by different concentrations of the compound of Comparative Example 2.

FIG. 22 shows the degrees of inhibition of T cell division by different concentrations of the compound of Comparative Example 3.

FIG. 23 shows the levels of IL-2, IL-4, IL-13 and TNF gene expression by the compound of Example 2, which were represented using the numbers of fragments per kilobase of transcript per million fragments after 1 h and 4 h.

FIG. 24 shows the levels of IL-3, NFATC1, REL, and MYC gene expression by the compound of Example 2, which were represented using the numbers of fragments per kilobase of transcript per million fragments after 1 h and 4 h.

FIG. 25 shows the degrees of inhibition of IL-2 production by different concentrations of the compound of Example 1.

FIG. 26 shows the degrees of inhibition of IL-2 production by different concentrations of the compound of Example 2.

FIG. 27 shows the degrees of inhibition of IL-2 production by different concentrations of the compound of Example 3.

FIG. 28 shows the degrees of inhibition of IL-2 production by different concentrations of the compound of Example 4.

FIG. 29 shows the degrees of inhibition of IL-2 production by different concentrations of the compound of Example 5.

FIG. 30 shows the degrees of inhibition of IL-2 production by different concentrations of the compound of Example 6.

FIG. 31 shows the degrees of inhibition of IL-2 production by different concentrations of the compound of Example 7.

FIG. 32 shows the degrees of inhibition of IL-2 production by different concentrations of the compound of Example 8.

FIG. 33 shows the degrees of inhibition of IL-2 production by different concentrations of the compound of Example 9.

FIG. 34 shows the degrees of inhibition of IL-2 production by different concentrations of the compound of Example 10.

FIG. 35 shows the degrees of inhibition of IL-2 production by different concentrations of the compound of Example 11.

FIG. 36 shows the degrees of inhibition of IL-2 production by different concentrations of the compound of Example 12.

FIG. 37 shows the degrees of inhibition of IL-2 production by different concentrations of the compound of Example 13.

FIG. 38 shows the degrees of inhibition of IL-2 production by different concentrations of the compound of Example 14.

FIG. 39 shows the degrees of inhibition of IL-2 production by different concentrations of the compound of Example 15.

FIG. 40 shows the degrees of inhibition of IL-2 production by different concentrations of the compound of Example 16.

FIG. 41 shows the degrees of inhibition of IL-2 production by different concentrations of the compound of Example 17.

FIG. 42 shows the degrees of inhibition of IL-2 production by different concentrations of the compound of Example 18.

FIG. 43 shows the degrees of inhibition of IL-2 production by different concentrations of the compound of Example 19.

FIG. 44 shows the degrees of inhibition of IL-2 production by different concentrations of the compound of Comparative Example 1.

FIG. 45 shows the degrees of inhibition of IL-2 production by different concentrations of the compound of Comparative Example 2.

FIG. 46 shows the degrees of inhibition of IL-2 production by different concentrations of the compound of Comparative Example 3.

FIGS. 47 and 48 are images showing the effects of the compounds of Examples 2, 3, and 6 on the prevention of rejection after transplantation.

FIG. 49 photographically shows the therapeutic effects of the compounds of Examples 14 and 17 on psoriasis.

FIGS. 50, 51, and 52 graphically show the therapeutic effects of the compounds of Examples 14 and 17 on psoriasis, which were evaluated by visual observation.

FIGS. 53, 54, and 55 are histopathological images (H&E) showing the therapeutic effects of the compounds of Examples 14 and 17 on psoriasis.

FIGS. 56 and 57 show changes of T cells in psoriasis-induced mice by the compounds of Examples 14 and 17.

FIG. 58 photographically shows the therapeutic effect of the compound of Example 2 on atopic dermatitis.

FIG. 59 shows histopathological images (H&E) showing the therapeutic effect of the compound of Example 2 on atopic dermatitis.

FIG. 60 shows histopathological images (toluidine blue staining) showing the therapeutic effect of the compound of Example 2 on atopic dermatitis.

FIG. 61 shows the therapeutic effect of the compound of Example 2 on atopic dermatitis based on an IgE level reduction.

### MODE FOR THE INVENTION

The present invention will now be described in more detail.

The present inventor has found that drugs capable of inhibiting the proliferation of T cells exert immunosuppressive activities and has succeeded in demonstrating the immunosuppressive activities through animal experiments using skin transplantation models as transplantation models, atopic dermatitis models as hypersensitive immune response model, and psoriasis models as autoimmune disease models. The present invention has been accomplished based on this finding.

The present invention will be more specifically explained with reference to the following examples. These examples are merely illustrative to assist in understanding the present invention and are not intended to limit the scope of the present invention.

These exemplary embodiments are provided to more fully explain the present invention to those skilled in the art.

Although the particulars of the present invention are described herein in detail, it will be obvious to those skilled in the art that such particulars are merely preferred embodiments and are not intended to limit the scope of the present invention.

Therefore, the true scope of the present invention is defined by the appended claims and their equivalents.

The present invention provides the use of a compound represented by Formula 1:

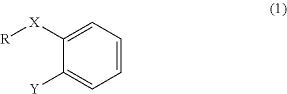

wherein R is a linear alkyl group, a branched alkyl group or an alkyl group containing one or more carbon-carbon double bonds, X is an amide or ester group, and Y is a halo, methoxy, ethoxy, hydroxyl or nitro group, in the preparation of an immunosuppressive medicament.

In the definition of R in Formula 1, the linear alkyl group has 6 to 14 carbon atoms and may be represented by CH₃(CH₂)ₙ— (wherein n is 6 to 14).

In the definition of R in Formula 1, the branched alkyl group may have a C₆ main chain and a C2 branch, represented by Structural Formula 1:

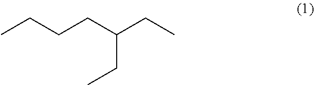

The number of carbon atoms in the main chain is not limited and may be in the range of 6 to 14.

In the definition of R in Formula 1, the alkyl group containing one or more carbon-carbon double bonds may have a C₈ main chain and two C1 branches, represented by Structural Formula 2:

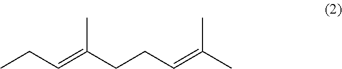

Here, the number of carbon atoms in the linear alkyl group, the branched alkyl group or the alkyl group containing one or more carbon-carbon double bonds is preferably 6 to 14, as mentioned for the branches and the main chains. If the number of carbon atoms is less than 6, the desired immunosuppressive effect of the compound is difficult to expect. Meanwhile, if the number of carbon atoms exceeds 14, the compound may be absorbed in vivo.

X in Formula 1 is the amide group represented by Structural Formula 3:

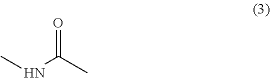

or the ester group represented by Structural Formula 4:

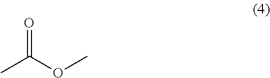

Y in Formula 1 is a halo, methoxy (MeO—), hydroxyl (OH—) or nitro (NO₂—) group. The halo group is fluoro (F—), chloro (Cl—), bromo (Br—) or iodo (I—).

The immunosuppressive effect of the compound can be further enhanced when X and Y in Formula 1 are in the ortho position.

Immunity is an important phenomenon necessary to protect the body against antigens such as pathogens. Immune responses are mediated by early innate and late adaptive immune responses. Innate immunity, also called native immunity, non-specific immunity, primary defense action or natural immunity, refers to an immune system that does not recognize and responds immediately to specific pathogens. Unlike innate immunity, adaptive immunity has exquisite specificities for different macromolecules and may cause very strong immune responses upon repeated exposure to the same antigens. The two types of adaptive immune responses are cell-mediated immunity and humoral immunity. Cell-mediated immunity is mediated by T lymphocytes.

T lymphocytes are divided into functionally distinct populations: helper CD4 (helper) T cells, including Th1 cells, Th2 cells, Th17 cells, and Treg cells; and CD8 cytotoxic T lymphocytes.

T cells recognize Class I and Class II MHC molecules in grafts with different genetic backgrounds after tissue or organ transplantation and cause specific immune responses, called rejection responses, that destroy the grafts. For successful organ or tissue transplantation, T cell-mediated immune responses need to be regulated.

For allergic diseases, some CD4+ helper T cells release IL-4 and IL-13 that stimulate isotype conversion of B-cell antibodies to immunoglobulin E (IgE). These T cells play a critical role in producing IgE, a special type of antibody that is very important in the pathogenesis of atopy. Thus, it is very important to regulate the immune responses of T cells.

Autoimmune diseases refer to diseases in which the immune system against foreign antigens such as pathogens recognizes its own organs or tissues as antigens of foreign origin to cause immune responses. T cells are known to be involved in the pathogenesis of autoimmune diseases. Such autoimmune diseases include rheumatoid arthritis, multiple sclerosis, inflammatory bowel disease, and psoriasis, whose mechanisms are known to be inflammation mediated by T-cell cytokines. For the treatment of these diseases, it is also very important to regulate the immune responses of T cells. Therefore, inhibition of T cell proliferation is of great importance to regulate the immune responses of T cells. The present inventor has found that drugs capable of inhibiting the proliferation of T cells exert immunosuppressive activities and has succeeded in demonstrating the immunosuppressive activities through animal experiments using skin transplantation models as transplantation models, atopic dermatitis models as hypersensitive immune response model, and psoriasis models as autoimmune disease models. The present invention has been accomplished based on this finding.

The medicament prepared using the compound of Formula 1 is suitable to inhibit cell-mediated immunity.

The medicament is suitable to treat allograft rejection.

The medicament is suitable to treat autoimmune diseases.

The medicament is suitable to treat psoriasis.

The medicament is suitable to treat atopy.

The medicament is suitable to treat insulin-dependent diabetes mellitus.

The medicament is suitable to treat multiple sclerosis.

The medicament is suitable to treat rheumatoid arthritis.

The immunosuppressive activity of the compound of Formula 1 can be demonstrated through T cell proliferation inhibition, IL-2 gene expression level measurement, and IL-2 protein expression level measurement tests.

The compound of Formula 1 with immunosuppressive activity can be used to prepare an immunosuppressive medicament that suppresses cell-mediated immunity and to treat diseases associated with altered immunologic adaptive responses, such as autoimmune diseases, allergic reactions, and “graft versus host” diseases with minimal side effects. Accordingly, the use of the compound of Formula 1 would help alleviate or cure the conditions of patients suffering from related diseases. Particularly, the compound of Formula 1 would also be useful in a prophylactic treatment of patients who received or are going to receive an allogeneic tissue or organ transplant, for preventing undesired immunologic reactions.

The present invention can be applied to mammals, including humans, who suffer from diseases such as autoimmune diseases or “graft versus host” diseases or are at risk of rejection against transplanted allogeneic tissues or organs. Examples of such mammals include household pets, mice, and rats.

An effective immunosuppressive amount of the compound of Formula 1 is that amount which is effective in providing an immunosuppressive effect. The immunosuppressive effect refers to the slowing, interrupting, inhibiting or preventing the further expression of the immune response or of the cell-mediated immune response.

The effective amount of the compound of Formula I can be readily determined by the attending diagnostician, as one skilled in the art, by the use of known techniques and by observing results obtained under analogous circumstances. In determining the effective amount or dose, a number factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; and other relevant circumstances.

The medicament including the compound of Formula 1 can be administered in any form or mode which makes the compound bioavailable in effective amounts, including oral and parenteral routes. For example, it can be administered orally, subcutaneously, intramuscularly, intravenously, transdermally, intranasally or rectally. Oral administration is preferred.

The medicament can also be used in combination with one or more other immunosuppressants to minimize the side effects of other drugs or to enhance the effects of other drugs.

Those skilled in the art of preparing formulations can readily select the proper form and mode of administration depending upon the particular characteristics of the compound selected, the disease state to be treated, the stage of disease, and other relevant circumstances.

The compound can be administered alone or in the form of a pharmaceutical composition in combination with pharmaceutically acceptable carriers or excipients, the proportion and nature of which are determined by the solubility and chemical properties of the compound selected and standard pharmaceutical practice.

The pharmaceutical composition is prepared in a manner well known in the pharmaceutical art. The carrier or excipient may be a solid, semi-solid, or liquid material which can serve as a vehicle or medium for the active ingredient. Suitable carriers or excipients are well known in the art.

The medicament may be adapted for oral or parenteral use and may be administered to the patient in the form of tablets, capsules, suppositories, solution, suspensions, or the like. The compound may be administered orally, for example, with an inert diluent or with an edible carrier. The compound may be enclosed in gelatin capsules or compressed into tablets.

For the purpose of oral therapeutic administration, the compound may be incorporated with excipients and used in the form of tablets, troches, capsules, elixirs, suspensions, syrups, wafers, chewing gums and the like.

The amount of the compound present in the medicament is such that a suitable dosage will be obtained.

The tablets, pills, capsules, troches and the like may also contain one or more of the following adjuvants: binders such as microcrystalline cellulose, gum tragacanth or gelatin; excipients such as starch or lactose; disintegrating agents such as alginic acid, Primogel™, corn starch and the like; lubricants such as magnesium stearate or Sterotex™; glidants such as colloidal silicon dioxide; and sweetening agents such as sucrose or saccharin may be added or a flavoring agent such as peppermint, methyl salicylate or orange flavor may be added. When the dosage unit form is a capsule, it may contain, in addition to materials of the above type, a liquid carrier such as polyethylene glycol or a fatty oil.

Other dosage unit forms may contain other various materials which modify the physical form of the dosage unit, for example, as coatings. Thus, tablets or pills may be coated with sugar, shellac, or other enteric coating agents. A syrup may contain, in addition to the present compound, sucrose as a sweetening agent and certain preservatives, dyes and colorings and flavors.

For the purpose of parenteral therapeutic administration, the compound of Formula 1 may be incorporated into a solution or suspension. The amount of the compound is such that a suitable dosage will be obtained.

The solutions or suspensions may also include the one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates; and agents for the adjustment of tonicity such as sodium chloride or dextrose. The parenteral preparation can be enclosed in ampules, disposable syringes or multiple dose vials made of glass or plastic.

### Example 1

The compound of Formula 2 was mixed with DMSO to prepare 10 μM, 20 μM, 30 μM, and 40 μM mixtures. The mixtures were dispensed into wells.

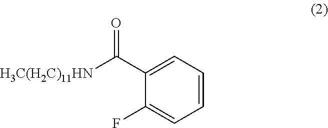

### Example 2

The procedure of Example 1 was repeated except that the compound of Formula 3 was used.

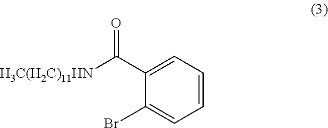

### Example 3

The procedure of Example 1 was repeated except that the compound of Formula 4 was used.

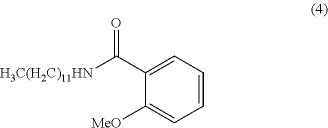

### Example 4

The procedure of Example 1 was repeated except that the compound of Formula 5 was used.

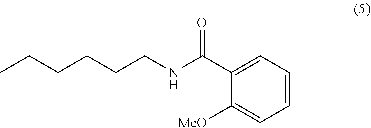 [SMILES: CCCCCCNC(=O)c1ccccc1OC]

### Example 5

The procedure of Example 1 was repeated except that the compound of Formula 6 was used.

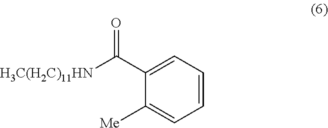

### Example 6

The procedure of Example 1 was repeated except that the compound of Formula 7 was used.

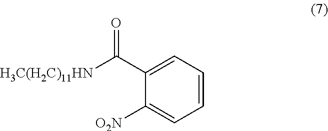

### Example 7

The procedure of Example 1 was repeated except that the compound of Formula 8 was used.

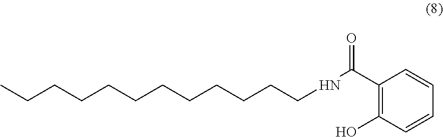 [SMILES: CCCCCCCCCCCCNC(=O)c1ccccc1O]

### Example 8

The procedure of Example 1 was repeated except that the compound of Formula 9 was used.

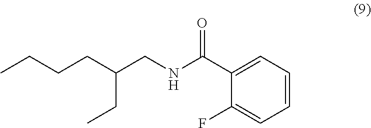 [SMILES: CCCCC(CC)CNC(=O)c1ccccc1F]

### Example 9

The procedure of Example 1 was repeated except that the compound of Formula 10 was used.

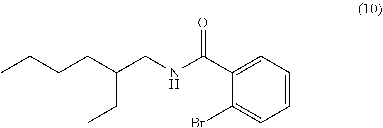 [SMILES: CCCCC(CC)CNC(=O)c1ccccc1Br]

### Example 10

The procedure of Example 1 was repeated except that the compound of Formula 11 was used.

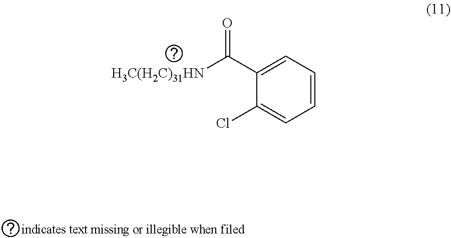

### Example 11

The procedure of Example 1 was repeated except that the compound of Formula 12 was used.

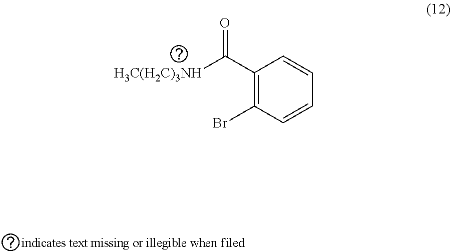

### Example 12

The procedure of Example 1 was repeated except that the compound of Formula 13 was used.

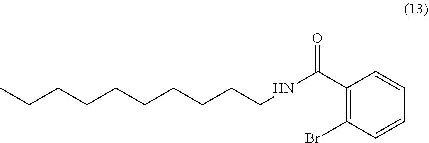 [SMILES: CCCCCCCCCCNC(=O)c1ccccc1Br]

### Example 13

The procedure of Example 1 was repeated except that the compound of Formula 14 was used.

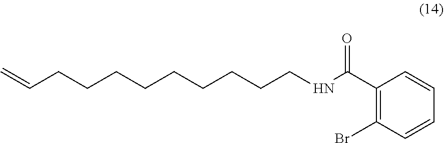 [SMILES: C=CCCCCCCCCCNC(=O)c1ccccc1Br]

### Example 14

The procedure of Example 1 was repeated except that the compound of Formula 15 was used.

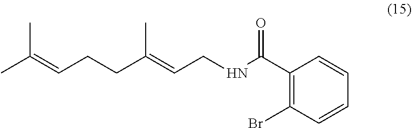 [SMILES: CC(C)=CCC/C(C)=C/CNC(=O)c1ccccc1Br]

### Example 15

The procedure of Example 1 was repeated except that the compound of Formula 16 was used.

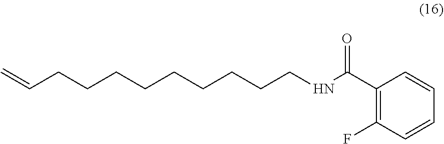 [SMILES: C=CCCCCCCCCCNC(=O)c1ccccc1F]

### Example 16

The procedure of Example 1 was repeated except that the compound of Formula 17 was used.

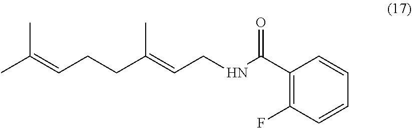 [SMILES: CC(C)=CCC/C(C)=C/CNC(=O)c1ccccc1F]

### Example 17

The procedure of Example 1 was repeated except that the compound of Formula 18 was used.

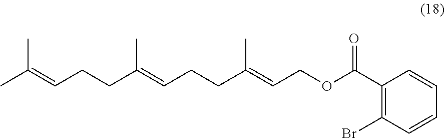 [SMILES: CC(C)=CCC/C(C)=C/CC/C(C)=C/COC(=O)c1ccccc1Br]

### Example 18

The procedure of Example 1 was repeated except that the compound of Formula 19 was used.

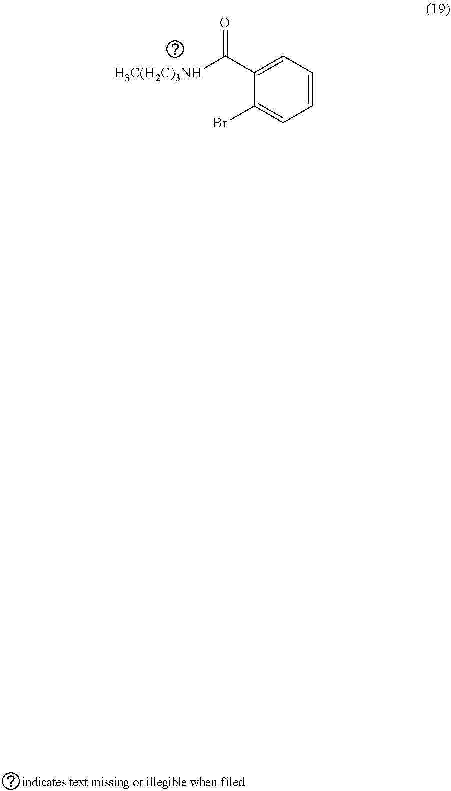

### Example 19

The procedure of Example 1 was repeated except that the compound of Formula 20 was used.

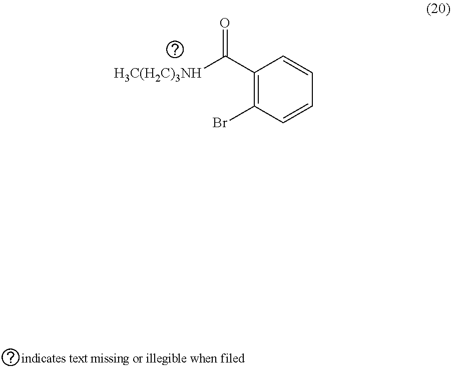

### Comparative Example 1

The procedure of Example 1 was repeated except that the compound of Formula 21 was used.

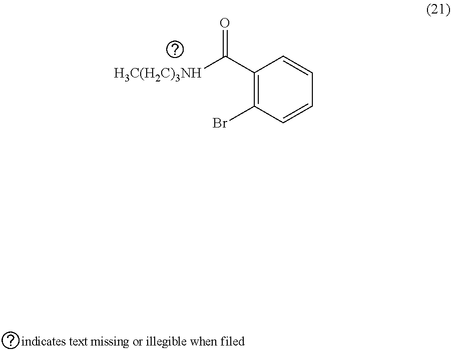

### Comparative Example 2

The procedure of Example 1 was repeated except that the compound of Formula 22 was used.

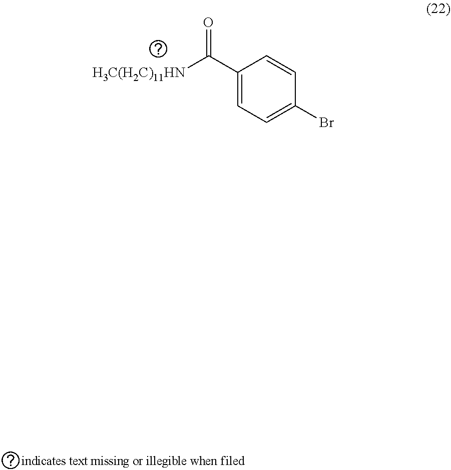

### Comparative Example 3

The procedure of Example 1 was repeated except that the compound of Formula 23 was used.

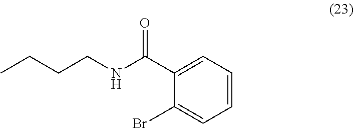

### Test Example 1: T Cell Proliferation Inhibition

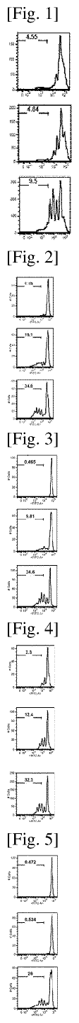

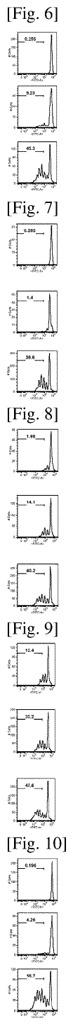

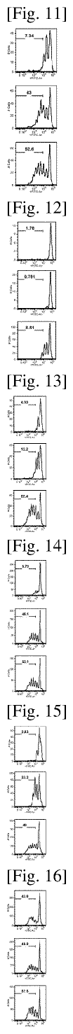

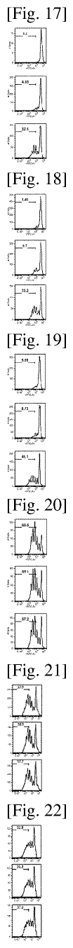

To investigate the inhibitory effects of the compounds of Examples 1-19 and Comparative Examples 1-3 on T cell proliferation, T cell proliferation was measured ex vivo using carboxyfluorescein diacetate succinimidyl ester (CFSE), a fluorescent dye that is covalently bonded to intracellular molecules. The spleens were harvested from 7-week-old C57BL/6 mice and crushed. Only single cells were isolated using a strainer (40 μm pore size), erythrocytes were removed with ammonium-chloride-potassium (ACK) lysis buffer, leaving only leukocytes, and CD 90.2 microbeads (miltenyi 130-121-278) were added thereto. After incubation at 4° C. for 20 min, only splenic T cells were isolated using a MACS magnetic stand and LS columns. The splenic T cells were suspended in 1 ml of free media (RPMI1640+200 U/mL penicillin+200 μg/mL streptomycin), 0.3 μl of CFSE (10 mM) was added thereto, followed by incubation at 37° C. for 5 min. The incubation was stopped quenched by addition of 10 ml of free media, followed by centrifugation to obtain a cell precipitate. RPMI 1640 medium supplemented with 10% FBS, 100 U/mL penicillin, and 100 μg/mL streptomycin was added to the cell precipitate to suspend the cells. The cell suspension was dispensed into the wells of a 96-well plate at a density of 2×10⁵ cells/well and treated with CD3 and CD28 antibodies (each 0.5 μg/ml) to activate T cells. Test results were acquired using a FACS Canto and are shown in FIGS. 1-22.

Referring to these figures, the concentration increases from the bottom graph to the top graph: 10 μM, 20 μM, and 30 μM. The compounds of Examples 1-19 exerted superior inhibitory effects on T cell proliferation, whereas the compounds of Comparative Examples 1-3 had insignificant inhibitory effects on T cell proliferation.

In each graph, the y-axis represents the amount (number) of cells and the x-axis represents the amount of CFSE in the cells. The fluorescence decreases as moving to the left of the graph. This decrease indicates the proliferation of cells.

### Test Example 2: Cytotoxicity

The cytotoxicities of the compounds of Examples 1-19 and Comparative Examples 1-3 to T lymphocyte cell line Jurkat E6-1 provided from the Korea Cell Line Bank were measured using a cell counting kit (CCK-8, Dojindo). After the cell line was allowed to grow in RPMI1640 medium supplemented with 10% FBS, 100 U/mL penicillin, and 100 μg/mL streptomycin, cells were plated in each well of a 96-well plate at a density of 2λ10⁴ cells/well and treated with each of the compounds of Examples 1-19 and Comparative Examples 1-3. After culture in a 5% CO₂ incubator at 37° C. for 20 h, CCK-8 solution was added to a concentration of 10%, followed by reculture in a 5% CO2 incubator at 37° C. for one and a half hours. The absorbance was measured at 450 nm (Reference: 650 nm) and was expressed in percent relative to the value *?* from non-treated cells (viability 100%). The results are shown in Table 1.

In Table 1, the number 100 corresponds to a viability of 100%.

TABLE 1

| | None (%) | 10 μM (%) | 20 μM (%) | 30 μM (%) |
|---|---|---|---|---|
| Example 1 | 100 | 97.3 | 94.3 | 96.6 |
| Example 2 | 100 | 99 | 96 | 89.8 |
| Example 3 | 100 | 98 | 94 | 90 |
| Example 4 | 100 | 96 | 92 | 88 |
| Example 5 | 100 | 88.7 | 87.2 | 85.7 |
| Example 6 | 100 | 99.3 | 99.2 | 99.4 |
| Example 7 | 100 | 82 | 79.3 | 66 |
| Example 8 | 100 | 94.7 | 92.2 | 90.9 |
| Example 9 | 100 | 95.3 | 95.1 | 92.0 |
| Example 10 | 100 | 98.7 | 96.8 | 91.8 |
| Example 11 | 100 | 89.8 | 91 | 93 |
| Example 12 | 100 | 91 | 85.3 | 78.5 |
| Example 13 | 100 | 98.8 | 97.9 | 90.3 |
| Example 14 | 100 | 96.9 | 96.6 | 95.4 |
| Example 15 | 100 | 88.8 | 91.2 | 93.5 |
| Example 16 | 100 | 89.3 | 85.7 | 76.3 |
| Example 17 | 100 | 73.6 | 72.1 | 72.7 |
| Example 18 | 100 | 80.2 | 79.3 | 75.7 |
| Example 19 | 100 | 88.4 | 90.5 | 94.2 |

As can be seen from the results in Table 1, the viabilities after treatment with the compounds of Examples 1-19 demonstrated very low cytotoxicities of the compounds.

### Test Example 3: Gene Expression Level Measurement

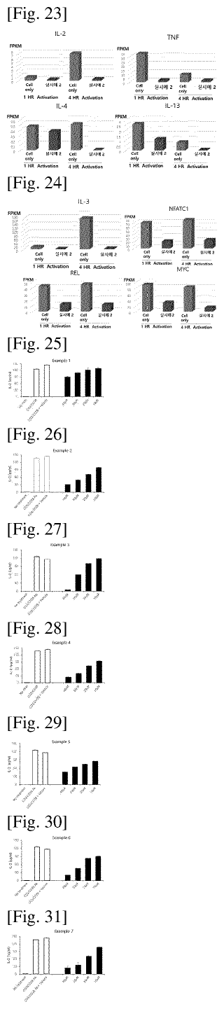

The levels of gene expression by the compound of Example 2 were measured to investigate how the inventive compound had an effect on the total gene expression in Jurkat T lymphocytes. To this end, 5×10⁶ Jurkat cells were non-treated or treated with the inventive compound for 30 min to prepare a non-treated cell culture sample and a treated cell culture sample, respectively. Phorbol 12-myristate 13-acetate (PMA) and ionomycin were added at concentrations of 25 ng/ml and 1 μM/mL, respectively, to each cell culture sample, followed by activation for 1 h or 4 h. The culture solution was centrifuged to obtain a cell precipitate. Trizol was added to the cell precipitate and total RNA was extracted according to the method recommended by the supplier. Genes involved in the activation of Jurkat T cells by the compound were discovered through RNA sequencing. The IL-2 gene is essential for the production of IL-2 protein, an important cytokine for inducing T-cell proliferation. The IL-4 gene is an essential gene for the production of IL-4 protein that induces the conversion of B progenitor cells into B cells capable of IgE and IgG4 production (“class switching”). The IL-2 gene promotes the production of MHC class II. The TNF gene is a cytokine gene called tumor necrosis factor (TNF) that causes an acute inflammatory response. The TNF gene is involved in the mechanisms of autoimmune diseases such as rheumatism. The IL-13 gene is a gene that is secreted by Th2 cells and mediates diseases such as allergic inflammation or asthma. The IL-13 gene is known to be produced from activated T cells when stimulated with IL-3, specific impulses or antigens. When the expression of IL-3, which is also known as a potent mast cell growth factor, is suppressed, the IL-13 gene inhibits mast cells and is thus effective against mast cell-mediated diseases such as atopy. The IL-13 gene can be used for myeloproliferative diseases and blood cancers because IL-3 is a hematopoietic factor necessary for the proliferation of hematopoietic progenitor cells. MYC lies at the crossroads of signal transduction pathways downstream of many ligand-membrane receptor complexes and responds immediately to regulate cell proliferation and growth. MYC originally acts as a helper that allows the division of stem cells such as embryonic cell to proceed more rapidly. MYC remains silent without being substantially expressed in normal cells that need not to grow or divide further. The Myc gene is located in 8924 and its overexpression is found in several carcinomas due to gene abnormalities such as amplification and translocation. It is known that p53 properly controls the functions of Myc in normal stem cells but the oncogene Myc gene maintains its high activity in cancer cells where Myc is overexpressed beyond the control level or p53 loses its function. Several experiments revealed that when the functions of Myc overexpressed in cancer cells are suppressed, the growth of cancer cells is inhibited, leading to death, and the resistance of cancer cells to existing anticancer drugs is reduced, creating a synergistic effect. For these reasons, control over the overexpression or activity of Myc has attracted attention as a promising approach to new anticancer drugs. Myc overexpression is found in various carcinomas, including most blood cancers. NFATc1 and Re1 are also known as important factors for cell proliferation. The graphs of FIGS. 23 and 24 confirm that the above-described genes were inhibited.

In conclusion, the compound of Example 2 can be used as a therapeutic agent for various types of carcinomas.

### Test Example 4: IL-2 Protein Expression Level Measurement

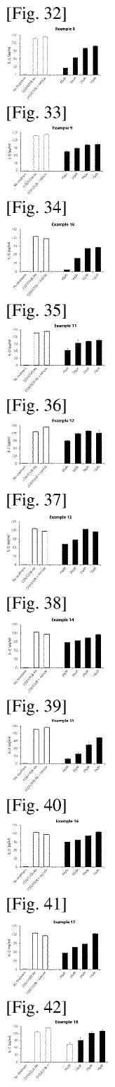

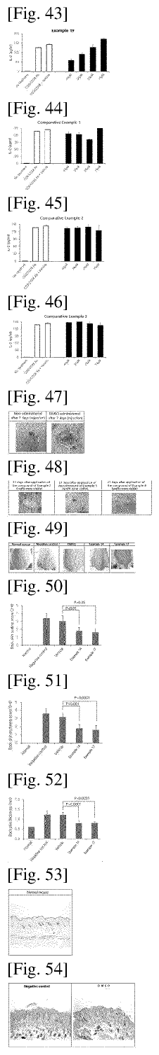

The spleens were excised from 7-week-old C57BL/6 mice and crushed. Only single cells were isolated using a strainer (40 μm pore size), erythrocytes were removed with ammonium-chloride-potassium (ACK) lysis buffer, leaving only leukocytes, and CD 90.2 microbeads (miltenyi 130-121-278) were added thereto. After incubation at 4° C. for 20 min, only splenic T cells were isolated using a MACS magnetic stand and LS columns. MACS buffer was removed by centrifugation to obtain a cell precipitate. RPMI 1640 medium supplemented with 10% FBS, 100 U/mL penicillin, and 100 μg/mL streptomycin was added to the cell precipitate to suspend the cells. The cell suspension was dispensed into the wells of a 96-well plate at a density of 2×10⁵ cells/well and treated with CD3 and CD28 antibodies (each 0.5 μg/ml) to activate T cells. Three wells were treated with each of the 10 μM, 20 μM, 30 μM, and 40 μM mixtures of the compounds of Examples 1-19. Cells were cultured in a 5% CO₂ incubator at 37° C. for 24 h. The soups were analyzed using ELISA kits for quantitative measurement of IL-2. The results are shown in FIGS. 25-43.

Referring to FIGS. 24-46, the administration of the inventive compounds was found to inhibit IL-2 production in a dose-dependent manner. The heights of the bars in the histograms represent the amounts of IL-2 produced, demonstrating that the inventive compounds inhibited IL-2 production. In contrast, the comparative compounds failed to inhibit T cell proliferation due to their inability to inhibit IL-2 production.

### Test Example 5: Transplantation

For skin transplantation experiments in animals, BALB/c mice (7 weeks old/20 g/female) as donors and C57/BL6 mice (7 weeks old/20 g/female) as recipients were purchased from Hana Biotech Co., Ltd., Korea. The animals were acclimated to the vivarium for a period of one week. After that, 100 mg/kg of alfaxan was administered intraperitoneally to each donor BALB/c mouse for anesthesia, the hair on the back of the mouse was shaved using a razor, the back was disinfected with 10% povidone, the skin of the back was removed to prepare grafts, and the grafts were stored on wet gauze in an ice Petri dish. 100 mg/kg of alfaxan was administered intraperitoneally to each recipient C57/BL6 mouse for anesthesia, the hair on the back of the mouse was shaved using a razor, the back was disinfected with 10% povidone, the skin (diameter 10 mm) of the back was removed with scissors while preserving the panniculus carnosus. The skin of the donor stored in the iced Petri dish was transplanted, a band was attached, and gentamycin was administered. The immunosuppressant was administered intraperitoneally to the recipient mouse twice a day at 12-hour intervals from two days before transplantation. During the administration, an observation was made as to whether rejection responses occurred after skin transplantation. The states of the grafts from mice not administered any drug after surgery and mice administered DMSO as a vehicle were observed and compared with those from the transplanted mice. The results are shown in FIGS. 47 and 48. Referring to FIGS. 47 and 48, rejection responses occurred in the naive mice and the mice administered the vehicle only on day 7 after surgery. In contrast, the grafts from the mice treated with the compounds of Examples 2, 3, and 6 were maintained for ≥21 days.

### Test Example 6: Autoimmune Diseases

A test was conducted to evaluate the alleviation of psoriatic skin lesions using psoriasis animals. To this end, first, BALB/c mice (7 weeks old/20 g/female) purchased from Hana Biotech Co., Ltd., Korea were acclimated to the vivarium for a period of one week. After that, the hair on the back of the mice was shaved and completely removed with a depilatory cream (Niclean cream (thioglycolic acid 80%, Ildong Pharmaceutical Co., Ltd., Korea)). The animals were bred for 24 h. 80 mg of Aldara® cream (imiquimod 4 mg) was applied daily for 1 week (7 days, 7 times in total) to induce psoriasis, as shown in FIG. 49. Thereafter, 80 mg of Aldara® cream (imiquimod 4 mg) was continuously applied to each group for 10 days. Each inventive compound was administered according to the schedule shown in Table 2. During the administration, an observation was made as to whether psoriasis was alleviated. As a result, skin lesions were alleviated in the group administered the inventive compound than in the negative control or the DMSO-administered group, as shown in FIG. 49.

Referring to FIG. 49, the skin lesions were alleviated in the group treated with the compound of Example 14 or 17.

TABLE 2

| | Topical | | |
|---|---|---|---|
| | application | | |
| | of imiquimod | | Number |
| Group | (4 mg) | Intervention | of mice |
| Normal | x | Shaving was only performed | 5 |
| | | (non-administered) | |
| Negative | ∘ | Shaving was only performed | 5 |
| control | | (non-administered) | |
| DMSO | ∘ | Intraperitoneal administration | 5 |
| (vehicle) | | of 50 μL of dimethyl | |
| | | sulfoxide (DMSO) | |
| Compound of | ∘ | Intraperitoneal administration | 5 |
| Example 14 | | of 50 μL of DMSO and 40 | |
| or 17 | | mg/kg of the compound | |

Referring to FIGS. 50, 51, and 52, the mice treated with the compounds of Examples 14 and 17 showed significant decreases in skin scaling, erythema, and thickness (P<0.05, P: P-value).

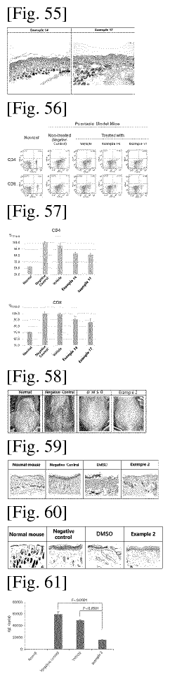

After the skin tissues of the test subjects were subjected to H&E staining according to the above schedule, the degrees of inflammatory cell infiltration were observed. The results are shown in FIGS. 53, 54, and 55. Severe hyperkeratosis, parakeratosis, and irregular acanthosis were observed in the group (negative control) applied with imiquimod only and the group applied with imiquimod and injected intraperitoneally with DMSO. In contrast, less inflammatory cell infiltration was observed in the mice administered with the compounds of Examples 14 and 17.

FIGS. 56 and 57 show the results of flow cytometry for the groups administered the compounds of Examples 14 and 17. The in vivo effects of the compounds of Examples 14 and 17 on T cells in the test subjects were investigated. To this end, the spleens were harvested from the mice administered each compound and T cells were extracted from the harvested spleens. Flow cytometry (FACS) revealed decreases in the number of CD4 T cells and CD8 T cells in the mice administered each compound, demonstrating that the inventive compounds inhibit the in vivo proliferation of T cells.

### Test Example 7: Allergic Diseases

7-week-old male NC/Nga mice as atopic animals were purchased from the Central Institute for Experimental Animals (SLC, Japan). The animals were acclimated to the vivarium for a period of one week (room temperature 21-23° C., humidity 50-60%). The hair on the back of the mice was shaved (3×4 cm), completely removed with a depilatory cream (Niclean cream (thioglycolic acid 80%)), and left for 24 h. 100 mg of Biostir ointment was applied to the back of each mouse with a cotton swab to induce atopy. Subsequently, 150 μl of 4% SDS solution was applied to the application site and dried completely for ˜2-3 h. Thereafter, 100 mg of atopic dermatitis-inducing Biostir ointment was evenly spread on the back twice a week a total of 8 times (4 weeks) to induce atopy. The experimental animals were divided into 4 groups for efficacy testing, as shown in Table 3. Specifically, Biostir ointment was applied a total of 8 times to induce atopy and then further applied twice a week for 2 weeks.

TABLE 3

| | Topical | | |
|---|---|---|---|
| | application of | | Number |
| Group | Biostir oint | Intervention | of mice |
| Normal | x | Shaving was only performed | 5 |
| | | (non-administered) | |
| Negative | ∘ | Shaving was only performed | 5 |
| control | | (non-administered) | |
| DMSO | ∘ | Intraperitoneal administration | 5 |
| (vehicle) | | of 50 μL of DMSO (vehicle) | |
| Compound of | ∘ | Intraperitoneal administration | 5 |
| Example 2 | | of 50 μL of DMSO and 40 | |
| | | mg/kg of the compound | |

Referring to FIG. 58, lesions were alleviated by administration of the compound of Example 2.

The skin tissues of the animals were excised, followed by H&E staining. Referring to FIG. 59, surface erosion, fibrinosuppurative exudate, and severe inflammatory cell infiltration were observed in the mice induced with atopy only and the mice intraperitoneally administered with DMSO only but they were alleviated in the mice administered the compound 3.

The degree of atopic dermatitis severity can be evaluated by the amount of mast cells in the skin. To investigate the therapeutic effect of the compound of Example 2 on atopic dermatitis, the skin tissues of the animals were stained with toluidine blue to observe mast cells. The results are shown in FIG. 60. Referring to FIG. 60, the number of mast cells in the skin of the mice administered the compound of Example 2 was reduced compared to those of the negative control and the DMSO-administered group.

Two weeks after administration of the compound of Example 2, the amounts of IgE in blood drawn from the hearts of the mice were measured. The results were compared with those obtained from the other groups. Referring to FIG. 61, the concentration of IgE in the group administered the compound of Example 2 was lower than that in the DMSO-treated group.

1. Use of a compound represented by Formula 1: 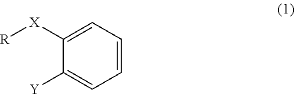
wherein R is a linear alkyl group, a branched alkyl group or an alkyl group containing one or more carbon-carbon double bonds, X is an amide or ester group, and Y is a halo, methoxy, ethoxy, hydroxyl or nitro group, in the preparation of an immunosuppressive medicament.

2. The use according to claim 1, wherein X and Y in Formula 1 are in the ortho position.

3. The use according to claim 1, wherein R in Formula 1 has 6 to 14 carbon atoms.

4. The use according to claim 1, wherein the medicament is useful for inhibiting cell-mediated immunity.

5. The use according to claim 1, wherein the medicament is useful for the treatment of allograft rejection.

6. The use according to claim 1, wherein the medicament is useful for the treatment of autoimmune diseases.

7. The use according to claim 1, wherein the medicament is useful for the treatment of cancer diseases.

8. The use according to claim 1, wherein the medicament is useful for the treatment of psoriasis.

9. The use according to claim 1, wherein the medicament is useful for the treatment of atopy.

10. The use according to claim 1, wherein the medicament is useful for the treatment of insulin-dependent diabetes mellitus.

11. The use according to claim 1, wherein the medicament is useful for the treatment of multiple sclerosis.

12. The use according to claim 1, wherein the medicament is useful for the treatment of rheumatoid arthritis.
